(19) United States
(12) Patent Application Publication
(10) Pub. No.: US 2008/0107758 A1
(43) Pub. Date: May 8, 2008

(54) TOPICAL STEROID SPRAY WITH BOTANIC SEED OILS

(75) Inventor: Charles E. Crutchfield III, Eagan, MN (US)

Correspondence Address: BRIGGS AND MORGAN P.A., 2200 IDS CENTER, 80 SOUTH 8TH ST, MINNEAPOLIS, MN, 55402, US

(73) Assignee: Cuticeuticals, Inc., St. Paul, MN (US)

(21) Appl. No.: 11/930,622

(22) Filed: Oct. 31, 2007

Related U.S. Application Data
(63) Continuation-in-part of application No. 10/462,458, filed on Jun. 16, 2003, now abandoned.
Application No. 10/462,458 is a continuation-in-part of application No. 09/882,965, filed on Jun. 15, 2001, now Pat. No. 6,579,512.

(51) Int. Cl. A61K 31/56; A61K 36/00; A61K 36/73; A61K 36/42; A61K 36/87; A61K 36/45; A61P 17/00

(52) U.S. Cl. 424/732; 514/171; 424/725; 424/765; 424/758; 424/766; 424/773

(57) ABSTRACT

A pharmaceutical topical spray composition of corticosteroid, an alcohol, a propellant, and botanic seed oils having a blend of black raspberry seed oil, black cumin seed oil, and red raspberry seed oil prepared according to a cold press method. A method for treating an inflammatory skin condition using the administration to the skin of a mammal of the pharmaceutical composition. The pharmaceutical composition is effective in the treatment of inflammatory skin conditions without the need for zinc pyrithione, undecylenic acid, or a detergent.

### CROSS REFERENCE TO RELATED APPLICATIONS

This application is a continuation-in-part of application Ser. No. 10/462,458, filed Jun. 16, 2003, currently pending and to be abandoned after entry of the instant application, which is a continuation-in-part of application Ser. No. 09/882,965, filed Jun. 15, 2001, entitled TOPICAL STEROID SPRAY, which issued on Jun. 17, 2003 under U.S. Pat. No. 6,579,512, the content of which is hereby incorporated by reference in their entirety.

### BACKGROUND OF THE INVENTION

The present invention relates to a pharmaceutical composition for the treatment of inflammatory skin conditions, and a therapeutic method for treating inflammatory skin conditions using the pharmaceutical composition.

Topical corticosteroids are powerful tools for treating skin disease. Understanding the correct use of these agents will result in the successful management of a variety of skin problems. There are many products available, and new ones appear almost monthly. Pharmaceutical companies have responded to the great demand for these agents with an increasing number of products, but all of these preparations have basically the same anti-inflammatory properties. They differ only in strength, base and price.

The anti-inflammatory properties of topical corticosteroids result in part from their ability to induce vasoconstriction of the small blood vessels in the upper dermis. This property is used in an assay procedure to determine the strength of each new product. These products are subsequently tabulated in seven groups, with group I the strongest and group VII the weakest (see the Formulary below).

| Group No. | Generic Name |
|---|---|
| I | Clobetasol propionate |
| II | Fluocinonide |
| III | Triamcinolone acetonide |
| IV | Fluocinolone acetonide |
| V | Hydrocortisone valerate |
| VI | Desonide |
| VII | Hydrocortisone |

This treatment recommends topical steroids by group number rather than by generic or brand name because the agents in each group are essentially equivalent in strength. When a new topical corticosteroid appears on the market, ask to which group it belongs and add it to the list in the Formulary.

Guidelines for choosing the appropriate strength of topical steroid are presented in the chart below.

SUGGESTED STRENGTH OF TOPICAL
STEROIDS TO INITIATE TREATMENT*

| GROUPS I-II | GROUPS III-V | GROUPS VI-VII |
|---|---|---|
| Psoriasis | Atopic dermatitis | Dermatitis (eyelids) |
| Lichen planus | Nummular eczema | Dermatitis (diaper area) |
| Discoid lupus† | Asteatotic eczema | Mild dermatitis (face) |
| Severe hand eczema | Stasis dermatitis | Mild anal inflammation |
| Poison ivy (severe) | Seborrheid dermatitis | Mild intertrigo |
| Lichen simplex chronicus | Lichen sclerosis et | |
| | atrophicus (vulva) | |
| Hyperkeratotic eczema | Intertrigo (brief course) | |
| Chapped feet | Tinea (brief course | |
| | to control inflammation) | |
| Lichen sclerosis et | Scabies (after scabicide) | |
| atrophicus (skin) | | |
| Alopecia areata | Intertrigo (severe cases) | |
| Nummular eczema (severe) | Anal inflammation | |
| | (severe cases) | |
| Atopic dermatitis | Severe dermatitis (face) | |
| (resistant adult cases) | | |

*Stop treatment, change to less potent agent, or use intermittent treatment once inflammation is controlled.
†Use on the face may be justified.

The best results are obtained when preparations of adequate strength are used for a specified length of time. Weaker, “safer” strengths often fail to provide adequate control. Patients who do not respond after 1 to 4 weeks of treatment should be reevaluated.

Additionally, topical preparations of the steroid clobetasol propionate are indicated for the relief of the inflammatory and pruritic manifestations of cortico-steroid-responsive dermatosis. See, for example, Maloney, et al., “Clobetasol Propionate Emollient 0.05% in the Treatment of Atopic Dermatitis”, International J. of Dermatology, 1998, 37, 128-144.

In the past, it has been found that clobetasol propionate is most effective in the treating of inflammatory skin conditions when combined with zinc pyrithione and undecylenic acid. For example, Seidel (U.S. Pat. No. 5,972,920) discloses the use of clobetasol propionate in combination with either zinc pyrithione, undecylenic acid, or both. Applicant Crutchfield also noted the requirement for zinc pyrithione in Crutchfield, et. al., “The Effective Use of Topical Zinc Pyrithione in the Treatment of Psoriasis: a Report of Three Cases”, J. Geriatr. Dermatol. 1997; 5(1):21-4.

Surprisingly, the applicant has found that zinc pyrithione and undecylenic acid are not necessary for the optimal effectiveness of clobetasol propionate.

Studies have also indicated that some sort of surfactant, such as sodium lauryl sulfate, is necessary for the optimal effectiveness of clobetasol propionate, whether alone or combined with zinc pyrithione and undecylenic acid. Again, Seidel '920 discloses the use of an anionic surfactant (sodium lauryl sulfate) in conjunction with clobetasol propionate, zinc pyrithione, and undecylenic acid.

Surprisingly, the applicant has found that no surfactant is necessary for the optimal effectiveness of clobetasol propionate.

Applicants have also found that the composition is most effective and easily tolerated by patients when administered in a spray form by means of a propellant. In contrast, Seidel '920 teaches away from the use of a spray as being highly evaporative and producing a painful freezing sensation to the skin and that some propellants are explosive.

### SUMMARY OF THE INVENTION

A pharmaceutical topical spray composition of corticosteroid, an alcohol, a propellant, and botanic seed oils including a blend of black raspberry seed oil, black cumin seed oil, and red raspberry seed oil. A method for treating an inflammatory skin condition using the administration to the skin of a mammal of the pharmaceutical composition of corticosteroid, an alcohol, a propellant, and botanic seed oils including a blend of black raspberry seed oil, black cumin seed oil and red raspberry seed oil. The pharmaceutical composition is effective in the treatment of inflammatory skin conditions without the need for zinc pyrithione, undecylenic acid, or a detergent.

### DETAILED DESCRIPTION OF THE PREFERRED EMBODIMENT

A first pharmaceutical composition for the treatment of inflammatory skin conditions consists essentially of clobetasol propionate, an alcohol, a propellant, and isopropyl palmitate, suitable for topical administration. A second pharmaceutical composition for the treatment of inflammatory skin conditions consists essentially of clobetasol propionate, an alcohol, a propellant, and a blend of black raspberry and black cumin seed oils, sold under the trademark Immuno-Viva®, a trademark owned by Botanic Oil Innovations, Inc., and further blended with red raspberry seed oil, and suitable for topical administration. The second pharmaceutical composition of clobetasol propionate, an alcohol, a propellant, and botanic seed oils including a blend of black raspberry seed oil, black cumin seed oil, and red raspberry seed oil, is suitably carried in a aerosol can with a nozzle. Applicants have found that no other active ingredients are necessary for the optimal pharmaceutical action of clobetasol propionate.

Starting with the first pharmaceutical composition, preferably, the clobetasol propionate is present in about 0.01 to 10% (% w/w). More preferably, the clobetasol propionate is present in about 0.01% to 1% (% w/w). Most preferably, the clobetasol propionate is present in the amount of 0.05% (% w/w). Any of the above corticosteroids may be used in this spray formulary.

Any number of alcohols can be used as a solvent in the preparation, such as methanol, ethanol, propanol, isopropanol, butanol, or isobutanol. However, Applicants have found that denatured ethanol (SDA-40 200 proof) is a suitable and effective alcohol to use in the composition. Most preferably, the ethanol is present in the amount of 37.43% (% w/w). However, a range of 27% (% w/w) to 47% (% w/w) may be used while a narrower range of 32% (% w/w) to 42% (% w/w) is more suitable.

The composition also contains isopropyl palmitate as an emollient oil or carrier most preferably in the amount of 37.72% (% w/w). However, a range of 27% (% w/w) to 47% (% w/w) may be used while a narrower range of 32% (% w/w) to 42% (% w/w) is more suitable. The alcohol and oil is about a 50:50 mixed blend.

An inactive ingredient, but one of importance in delivery of the composition to the skin, is a propellant. Any propellant conventionally used in the delivery of aerosol sprays may be used. Most preferably, an amount of 24.51% (% w/w) of isobutane is in the composition. However, a range of 20% (% w/w) to 30% (% w/w) may also be suitable.

A small amount of water, approximately 0.22%, is appropriate.

Importantly, the composition does not contain either zinc pyrithione or undecylenic acid.

A therapeutic method for treating an inflammatory skin condition comprises administering the above first composition to the skin of a mammal in need of such therapy.

The second pharmaceutical composition for the treatment of inflammatory skin conditions consists of clobetasol propionate, an alcohol, a propellant, and botanic seed oils including a blend of black raspberry oil, black cumin seed oil, and red raspberry seed oil, suitable for topical administration.

The second pharmaceutical composition differs significantly from the first pharmaceutical composition by using concentrated plant seed oils, also referred to as botanic seed oils prepared according to a cold press method, as the emollient oil or vehicle. Plant seed oils are an excellent source of antioxidants. In addition to traditional antioxidants, such as vitamins C and E, plant seed oils contain phenolic compounds which are excellent free radical scavengers. Black raspberry and red raspberry seed oils have a diversity and ultra-rich content of antioxidants, including 4 different forms of Vitamin E (Alpha and Gamma Tocopherol, Beta and Gamma Tocotrienol). These raspberry seed oils contain Omega 3 and Omega 6. In U.S. Patent Publication No. 20070243310, Synergistic super potent antioxidant cold pressed botanic oil blends, to Leonard et al., published Oct. 18, 2007, the inventors describe blends of seed oils as having a synergistic antioxidant effect.

Concentrated seed oils are described as immunostimulants in U.S. Patent Publication No. 2007/0128301, Immune Enhancement by Seed Oil and/Or Seed Flour, to Saltzman et al., published Jun. 7, 2007, and for use in treatment of cancer in Patent Publication No. 2005/0244375, Composition and Method of Cancer Treatment, to Leonard et al., published Nov. 3, 2005. In Saltzman et al., the applicant theorized that concentrated seed oils possess anti-inflammatory properties.

The use of concentrated seed oils in the pharmaceutical composition acts as natural emollients to prevent dryness and protect the skin, acting as a barrier and healing agent, as well as a soothing and softening agent of the skin. The concentrated seed oils in the pharmaceutical composition can reduce roughness, cracking and irritation. The use of seed oil emollients nourish the skin with concentrated nutrients that can be beneficial in treating inflammatory conditions of the skin such as acne, psoriasis, eczema and rosacea. The antioxidant properties of botanic seed oils may prove to have some of the most effective rejuvenating properties of any known skin treatment to date. Charles E. Crutchfield, M.D. Assoc. Prof. of Dermatology, University of Minnesota, et al., The Use of Nature Fresh Cold Pressed Seed Oils for Skin and Personal Care Products A New Approach (an article pending publication).

In The Use of Nature Fresh Cold Pressed Seed Oils for Skin and Personal Care Products—A New Approach article, there is further discussion about research showing that synergistic botanic seed oils can inhibit Cox-2 activity. Cox-2 is an enzyme linked to inflammation and inflammation is associated with many skin disorders ranging from sunburn to roseacea, psoriasis, acne and dandruff. In addition, the article discusses research that synergistic botanic seed oils have potent antimicrobial activity. Many skin disorders are known to result from or be exacerbated by bacteria and fungi living on the skin surface.

A preferred method of preparing the seed oils, used in the invention, is described in U.S. Patent Publication No. 2007/0128301, Immune Enhancement by Seed Oil and/Or Seed Flour, Saltzman et al, published Jun. 7, 2007, U.S. Patent publication is incorporated by reference herein in its entirety. That description is:

“The oils for the composition are prepared from seeds which have been carefully dried and cleaned at temperatures below 120 degrees F. In a cold press process, the seeds are fed through the press and put under high pressure with no extra heat during the pressing process. Oil temperatures during extraction are typically 70 degrees to 90 degrees F. To insure minimal or no oxidation and the highest potential antioxidant level of the oils, the press head and oil extraction chamber can be enclosed within an inert atmosphere. Refining or removal of suspended solids and container filling can also be done in an inert atmosphere to preserve quality.”

U.S. Patent Publication No. 2007/0128301, paragraph [0016].

Preferably, in the second pharmaceutical composition the clobetasol propionate is present in about 0.01 to 10% (% w/w). More preferably, the clobetasol propionate is present in about 0.01% to 1% (% w/w). Most preferably, the clobetasol propionate is present in the amount of 0.05% (% w/w). Any of the above corticosteroids may be used in this spray formulary.

As discussed above, clobetasol propionate, a Group I topical corticosteroids, is a preferred steroid. However, weaker topical corticosteroids in Groups II through VII can be used in the spray formulary with an adjustment in the weight for a therapeutically effective replacement for clobetasol propionate. This involves increasing the topical steroid amount to compensate for weaker activity. Whereas, the clobetasol propionate is more preferably present in about 0.01% to 1% (% w/w), the weaker topical corticosteroids in Groups II through VII can be used more preferably in an amount of about 0.01% to 2.5% (% w/w). The increase in the amount of the topical corticosteroid would result in a decrease in the amount of emollient seed oils. A person of skill in the art would be able to determine the appropriate effective therapeutic amount for replacement with another topical steroid of different strength.

Any number of alcohols can be used as a solvent in the preparation, such as methanol, ethanol, propanol, isopropanol, butanol, or isobutanol. However, Applicants have found that denatured ethanol (SDA-40 200 proof) is a suitable and effective alcohol to use in the composition. Most preferably, the ethanol is present in the amount of 37.43% (% w/w). However, a range of 27% (% w/w) to 47% (% w/w) may be used while a narrower range of 32% (% w/w) to 42% (% w/w) is more suitable.

The second pharmaceutical composition contains botanic seed oils including a blend of black raspberry seed oil, black cumin seed oils, and red raspberry seed oil as an emollient oil or carrier. The blend of black raspberry oil and black cumin seed oil sold as Immuno-Viva® has a ratio of about 50:50.

An embodiment of the second pharmaceutical composition used to treat patients with psoriasis employed a blend of botanic seed oils is in the amount of about 8.96% (% w/w) black raspberry seed oil, about 8.96% (% w/w) black cumin seed oil, about 17.92% (% w/w) red raspberry seed oil, 0.27% (% w/w) pumpkin seed oil, 0.27% (% w/w) chardonnay grape seed oil, 0.27% (% w/w) carrot seed oil, 0.27% (% w/w) blueberry seed oil, 0.27% (% w/w) cranberry seed oil, 0.27% (% w/w) pomegranate seed oil that makes up a total seed oil blend of about 37.72% (% w/w). The ratio of the blend of black raspberry and black cumin seed oils, sold under Immuno-Viva®, to the red raspberry oil was about 47.5:47.5. It is preferable that the ratio of black raspberry and black cumin seed oils to red raspberry seed oil be about 47.5 to 47.5, and this portion of the seed oil blend be about 36% (% w/w). The other amounts of seed oil, about 2% (% w/w), make up the remainder of the complete blend to be about 38% (% w/w).

A range of 27% (% w/w) to 47% (% w/w) of the complete blend of seed oils in the above proportions can be used, while a narrower range of 32% (% w/w) to 42% (% w/w) in the above proportions is more suitable.

Blends of two or more of the concentrated seed oils are preferred to use of a single seed oil. A preferred composition involves a blends consisting essentially of three concentrated seed oils in the pharmaceutical composition as the predominant component of this seed oil blend having synergistic antioxidant properties.

Not to be bound by theory, but blends of concentrated seed oils in the present invention are theorized to work with topical steroids such as clobetasol proprionate, to increase the healing effectiveness of the topical steroid when used on inflammatory skin conditions.

An inactive ingredient, but one of importance in delivery of the composition to the skin, is a propellant. Any propellant conventionally used in the delivery of aerosol sprays may be used. Most preferably, an amount of 24.51% (% w/w) of isobutane is in the composition. However, a range of 20% (% w/w) to 30% (% w/w) may also be suitable.

A small amount of water, approximately 0.22%, is appropriate.

The composition does not contain either zinc pyrithione or undecylenic acid.

Under the inventor's supervision, 1,450 patients with an inflammatory skin condition, psoriasis, were treated with a preferred embodiment of the second pharmaceutical composition having clobetasol propionate, an alcohol, a propellant, and botanic seed oils in a blend of black raspberry seed oil, black cumin seed oils, red raspberry seed oil, pumpkin seed oil, chardonnay grape seed oil, carrot seed oil, blueberry seed oil, cranberry seed oil, pomegranate seed oil. The results showed that 96% of the patents reported significant improvement using the second pharmaceutical composition.

A therapeutic method for treating an inflammatory skin condition comprises administering the second pharmaceutical composition to the skin of a mammal in need of such therapy.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

What is claimed:

1. A therapeutic method for treating an inflammatory skin condition comprising administering by spray to the skin of a mammal in need of such therapy, an effective amount of a pharmaceutical composition consisting essentially of a topical corticosteroid and a blend of three or more botanic seed oils that are prepared according to a cold press method.

2. The therapeutic method of claim 1, wherein the topical corticosteroid is present in about 0.01% to 2.5% (% w/w), and selected from the group consisting of clobetasol propionate, fluocinonide, triamcinolone acetonide, fluocinolene acetonide, hydrocortisone valerate, desonide, and hydrocortisone

3. The therapeutic method of claim 1, wherein the blend of three or more botanic seed oils is present in a range of 27% (w/w) to 47% (% w/w), and the blend of botanic seed oils comprising three or more plant seed oils selected from black cumin, black raspberry, red raspberry, pumpkin seed oil, chardonnay grape seed oil, carrot seed oil, blueberry seed oil, cranberry seed oil, and pomegranate seed oil.

4. The therapeutic method of claim 1, wherein the blend of three or more botanic seed oils is present in a range of 32% (w/w) to 42% (% w/w).

5. The therapeutic method of claim 1, wherein the inflammatory skin condition is psoriasis, atopic dermatitis, eczema, lupus, poison ivy, scabies, severe skin inflammation, dermatitis, lichen, or papulosquamous.

6. The therapeutic method of claim 1, wherein the pharmaceutical composition is delivered in spray form by means of a propellant comprising approximately 25% (% w/w) of isobutane.

7. The therapeutic method of claim 1, wherein the pharmaceutical composition is delivered in spray form by means of isobutane in a range of 20% (%/ow/w) to 30% (% w/w).

8. The therapeutic method of claim 1, wherein the administration is performed in the absence of zinc pyrithione and undecylinic acid.

9. A pharmaceutical topical spray composition consisting essentially of clobetasol propionate and a blend of black cumin seed oil, black raspberry seed oil, and red raspberry seed oil that are prepared according to a cold press method as a carrier.

10. The pharmaceutical composition of claim 9, wherein the clobetasol propionate is present in about 0.01 to 10% (% w/w).

11. The pharmaceutical composition of claim 9, wherein the clobetasol propionate is present in about 0.05% (% w/w).

12. The pharmaceutical composition of claim 9, wherein the composition is delivered in spray form by means of a propellant comprising 25% (% w/w) of isobutane.

13. The pharmaceutical composition of claim 9, wherein said composition is free of zinc pyrithione and undecylenic acid.

14. The pharmaceutical composition of claim 9, wherein the blend of black cumin seed oil, black raspberry seed oil, and red raspberry seed oil is present in a range of 27% (% w/w) to 47% (% w/w).

15. A pharmaceutical topical spray composition consisting essentially of clobetasol propionate in a range of about 0.01% to 1% (% w/w) and a blend of black cumin seed oil, black raspberry seed oil, and red raspberry seed oil, prepared according to a cold press method, in a range of 32% (% w/w) to 42% (% w/w).

16. The pharmaceutical composition of claim 15, wherein the blend of black cumin seed oil, black raspberry seed oil, and red raspberry seed oil is present in about 36% (% w/w).

17. The pharmaceutical composition of claim 15, wherein the clobetasol propionate is present in about 0.05% (% w/w).

18. A therapeutic method for treating an inflammatory skin condition comprising administering by spray to the skin of a mammal in need of such therapy, an effective amount of a pharmaceutical composition consisting essentially of clobetasol propionate, a blend of black cumin seed oil, black raspberry seed oil, and red raspberry seed oil that are prepared according to a cold press method.

19. The therapeutic method of claim 18, wherein the blend of black cumin seed oil, black raspberry seed oil, and red raspberry seed oil is present in a range of 32% (w/w) to 42% (% w/w).

20. The therapeutic method of claim 18, wherein the blend of black cumin seed oil, black raspberry seed oil, and red raspberry seed oil is present in about 36% (% w/w).

21. The therapeutic method of claim 18, wherein the inflammatory skin condition is psoriasis, atopic dermatitis, dermatitis, lichen planus, or papulosquamous.

22. The therapeutic method of claim 18, wherein the administration is performed in the absence of zinc pyrithione.

23. A pharmaceutical topical spray composition comprising clobetasol propionate in a range of about 0.01 to 1% (% w/w), a blend of black cumin seed oil, black raspberry seed oil, red raspberry seed oil, pumpkin seed oil, chardonnay grape seed oil, carrot seed oil, blueberry seed oil, cranberry seed oil, and pomegranate seed oil in a range of about 32% (% w/w) to 42% (% w/w), and said seed oils prepared according to a cold press method.

24. The pharmaceutical composition of claim 23, wherein the blend of black cumin seed oil, black raspberry seed oil, red raspberry seed oil is about 36% (% w/w), the clobetasol propionate is about 0.05% (% w/w), and wherein the composition is delivered in spray form by means of a propellant comprising 25% (% w/w) of isobutane

25. The pharmaceutical composition of claim 23, wherein said composition is free of zinc pyrithione and undecylenic acid.
